(19) United States
(12) Patent Application Publication
(10) Pub. No.: US 2015/0164528 A1
(43) Pub. Date: Jun. 18, 2015

(54) TISSUE EXTRACTION DEVICES AND RELATED METHODS

(71) Applicant: Boston Scientific Scimed, Inc., Maple Grove, MN (US)

(72) Inventors: Kenneth P. REEVER, Hopedale, MA (US); Ronald CIULLA, Westford, MA (US)

(73) Assignee: Boston Scientific Scimed, Inc.

(21) Appl. No.: 14/556,358

(22) Filed: Dec. 1, 2014

Related U.S. Application Data
(60) Provisional application No. 61/915,295, filed on Dec. 12, 2013.

(51) Int. Cl. A61B 17/32; A61M 1/00; A61B 1/307; A61M 3/02

(52) U.S. Cl. CPC A61B 17/32002; A61M 3/0279; A61M 1/008; A61B 1/307; A61M 2210/166

(57) ABSTRACT

A medical device is disclosed which may include an elongate luminal element configured for insertion into a natural body lumen. An elongate working member may positioned within the elongate luminal element. The elongate luminal element may include at least a first lumen which may extend along a longitudinal axis of the multi-lumen element. The working member may include an auger portion on a distal end thereof which may be configured to pierce tissue. In addition, the auger portion may be rotatable about the longitudinal axis.

### CROSS REFERENCE TO RELATED APPLICATIONS

This application claims the benefit of U.S. Provisional Application No. 61/915,295, filed Dec. 12, 2013, the disclosure of which is incorporated herein by reference in its entirety.

### DESCRIPTION OF THE DISCLOSURE

1. Field of the Disclosure

Embodiments of the present disclosure relate generally to medical devices and procedures. In particular, embodiments of the present disclosure relate to medical devices and methods for tissue extraction.

2. Background of the Disclosure

Benign Prostatic Hyperplasia (BPH) is noncancerous enlargement of the prostate gland in men. BPH includes hyperplasia (an increase in the number of cells) of prostatic stromal and epithelial cells which result in the formation of large nodules in the periurethral region of the prostate. As the prostate enlarges it puts pressure on and/or partially or completely occludes the urethra. Additionally, prostate enlargement may cause pain, difficulty in urination, infection, or the like.

Holmium Laser Enucleation of the Prostate (HoLEP) is a technique for treating BPH. HoLEP typically involves inserting a laser device into the urethra and directing the device to a target tissue including enlarged prostate tissue. Typically, such laser devices are directed to the target tissue using a sheath such as, for example, a cystoscope and/or rectoscope. The laser device enucleates (e.g., separates or removes) the target prostate tissue away from its surroundings. Typically the separated prostate tissue may form one or more large pieces of tissue, referred to as “tissue balls”, which are then directed (e.g., pushed) into the bladder using the laser device or another medical device. While referred to herein as a “ball,” the severed tissue may not necessarily be in the shape of a ball (e.g., sphere) but rather, may have any shape including irregular shapes. The laser device is then removed and another device such as a morcellator is introduced into the sheath for removing the tissue. A morcellator is a surgical device having a small opening at its distal end, one or more cutting blades, and suction capability. The blades may cut (e.g., mince, puree) the large pieces of tissue, e.g., tissue balls, that were moved into the bladder into smaller pieces. These smaller pieces may then be removed out of the body through the opening via, suction and/or other means.

There are, however, disadvantages to using conventional methods for removing tissue balls. For example, one or more of the smaller pieces of tissue formed by the morcellator may evade removal and remain in the body, which may interfere with or block other systems in the body. In addition, due to its small opening at the distal end, the morcellator may require a significant amount of time to cut the larger pieces of tissue into smaller ones for removal from the body. Additionally, once in the bladder, the tissue balls are free floating and have a tendency to avoid capture by bouncing off the bladder wall and/or floating away from the morcellator. Thus, it becomes difficult to contact them for morcellation. Moreover, since the morcellator includes one or more moving blades, there is a risk of damaging surrounding tissue. Accordingly, it may be desirable to provide for alternative systems and methods for tissue extraction.

### SUMMARY OF THE DISCLOSURE

Embodiments of the present disclosure include a medical device, such as a tissue extractor, that may be used to remove excised tissue from a body and methods of use thereof.

In one exemplary embodiment, a medical device may include an elongate luminal element configured for insertion into a natural body lumen. An elongate working member may positioned within the elongate luminal element. The elongate luminal element may include at least a first lumen which may extend along a longitudinal axis of the multi-lumen element. The working member may include an auger portion on a distal end thereof which may be configured to pierce tissue. In addition, the auger portion may be rotatable about the longitudinal axis.

The medical device may further include one or more of the following features: a suction line in fluid communication with the first lumen, the suction line may be configured to urge tissue in the first lumen; wherein the elongate luminal element may include a second lumen which may be configured to receive an optical member therein; an irrigation line, which may be in fluid communication with the second lumen; the elongate member may include a first portion and a second portion, and wherein the first portion may be more flexible than the second portion; a handle portion which may include a grip; the first lumen may further include a tissue holder at a distal portion thereof, the tissue holder may extend radially inwardly toward the longitudinal axis; the auger portion may include at least a portion of a helical turn; the auger portion may include a sharpened edge which may be configured to urge tissue into the first lumen when auger portion is rotated; and at least one of a rectoscope or cystoscope may be configured to receive elongate luminal element therein.

In another exemplary embodiment, a method of removing excised tissue from a body of a patient is disclosed. The method may include inserting a medical device proximate to a target tissue. The medical device may include an elongate working member configured to be received within a first lumen of an elongate luminal element. The first lumen may extend along a longitudinal axis and the working member may include an auger portion at the distal end thereof. The method may additionally include piercing the target tissue region with the auger portion. In addition, the method may include rotating the auger portion so as to proximally urge the target tissue into the first lumen. Further, the method may include removing the medical device from the body of the patient.

The method may further include one or more following features: applying a suction force to the first lumen which may urge tissue into the first lumen; a tissue holder at the distal portion of the first lumen, which may extend radially inwardly toward the longitudinal axis, and which may be configured to assist in preventing tissue pierced by the auger portion from rotating within the first lumen; the auger portion may include at least a portion of a helical turn; and the elongate luminal element may include a second lumen configured to receive an optical member, where the method may further include irrigating the second lumen with an irrigation source.

In another exemplary embodiment, a medical device may include a delivery member, which may be configured for insertion into a natural body lumen. The medical device may also include a working member, which may be configured to be received within the first lumen of the delivery member. The first lumen may extend along a longitudinal axis. The working member may include an auger portion on the distal end thereof. In addition, the auger portion may include a sharpened distal tip configured to pierce tissue. Further, the auger portion may be rotatable about the longitudinal axis.

The medical device may further include one or more of the following features: a suction line may be in fluid communication with the first lumen, the suction line may be configured to urge tissue into the first lumen; the auger portion may include a sharpened edge which may be configured to urge tissue into the first lumen when the auger portion is rotated; the first lumen may further include a tissue holder at the distal portion thereof, the tissue holder may extend radially inwardly toward the longitudinal axis; and the delivery member may further include a second lumen configured to receive an optical member therein, and the device may further include an irrigation line in fluid communication with the second lumen.

Additional objects and advantages of the present disclosure will be set forth in part in the description which follows, and in part will be understood from the description, or may be learned by practice of the claimed disclosure. The objects and advantages of the claimed disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates a male pelvic region with a healthy urinary system;

FIG. 2 illustrates a male pelvic region suffering from Benign Prostatic Hyperplasia (BPH); and

FIG. 3 illustrates an exemplary medical device, according to an embodiment of the present disclosure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts. As used herein, the term “distal” refers to the direction that is away from the user and into the patient's body. By contrast, the term “proximal” refers to the direction that is closer to the user and away from the patient's body.

### Overview

Embodiments of the present disclosure may include a medical device for removing and/or retrieving a material from a body and methods of use thereof. In some embodiments, the medical device may be used to retrieve tissue that has been cut away or otherwise severed from its surroundings. In at least one embodiment, the tissue to be removed may be tissue from the prostate for treatment of BPH. In alternative embodiments, the medical device may be used to remove other types of tissues or materials such as, for example, bladder stones, kidney stones, and the like. For convenience, the exemplary medical device discussed herein is referred to as an extraction device; however, this reference is merely made for convenience, and is intended to include devices capable of other and/or additional operations and/or functions such as resection.

In the following sections, embodiments of the present disclosure will be described using the bladder as an exemplary body organ. It will be understood that the bladder is merely an example and that the device may be utilized in other parts of the body, for example the uterus or the intestines.

The present disclosure provides medical devices for removing tissue from a patient's body, such as, tissue extraction devices. The medical devices may be used to remove tissue that has been cut away or excised from the body. An opening or working channel of the extraction device may facilitate removal of excised tissue from the body. The extraction device may be configured to be introduced into the body through a suitable natural opening, such as through the urethra.

### Exemplary Embodiments

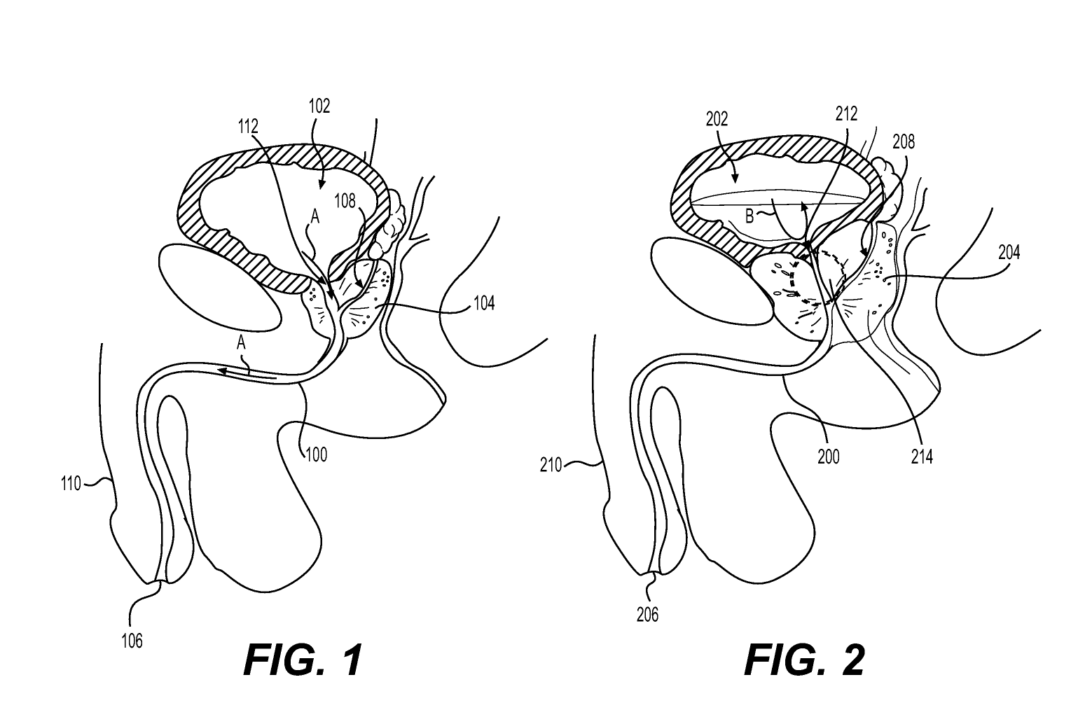

FIG. 1 illustrates the pelvic region of a male having a healthy urinary system. As shown in FIG. 1, the urinary system includes a urethra 100, a bladder 102, a prostate 104, a urinary meatus 106, and an ejaculatory duct 108 of the male urinary system. The urethra 100 is a biological lumen connecting bladder 102 to the urinary meatus 106 at the tip of the penis 110. The urethra 100 connects to the bladder 102 at bladder opening 112. As shown, the prostate 104 is positioned around the urethra 100 between the bladder 102 and the penis 110. Upon stimulation, the bladder 102 constricts and urine (depicted by arrow A) is released out of the urinary meatus 106 through the urethra 100.

FIG. 2 illustrates the male pelvic region suffering from Benign Prostate Hyperplasia (BPH). As shown, the pelvic region includes a urethra 200, a bladder 202, a prostate 204, a urinary meatus 206, an ejaculatory duct 208, a penis 210, and a bladder opening 212. In BPH, the passage of urine through the urethra 200 from the bladder 202 to the urinary meatus 206 at the tip of the penis 210 is obstructed by the enlarged prostate 204. For example, an excess tissue region 214 in the enlarged prostate 204 may constrict (e.g., narrow, obstruct, and/or partially occlude) the urethra 200 proximate to the bladder opening 212, and may cause pain, difficulty in urination, and/or urinary infections. For example, due to constriction of the urethra, urine (depicted by arrow B) is prevented from freely flowing through the urethra 200 to exit the penis 210 via the urinary meatus 206. In turn, it may result in urine buildup in the bladder 202. This accumulation of urine may increase infection and the occurrence of other urinary tract problems. In addition, the enlarged prostate 204 may constrict (e.g., narrow, obstruct, and/or partially occlude) the ejaculatory duct 208 causing erectile dysfunction or ejaculatory problems.

The enlarged prostate 204 may be severed using various methods, such as, laser enucleation as is known in the art. In laser enucleation, a laser is used to cut (e.g., sever, ablate) tissue from remaining portions of the enlarged prostate 204. By way of example only, laser enucleation may be used to cut excess tissue region 214 from the enlarged prostate 204 in order to alleviate constriction of the urethra 200. Cutting or severing of such tissue may form a tissue ball. Once formed, the tissue ball may be moved (e.g., pushed) into the bladder 202 for removal via an appropriate tool. The tissue ball is undesirable and may be removed using high-pulse energy to destroy the tissue ball, or by using a morcellating tool to cut the tissue ball into small pieces. However, removal of the small tissue pieces is difficult as they float freely in the urethra 200 and the bladder 202. Additionally, these small pieces may move to other body systems and cause unintentional problems. In addition, dissection of the tissue balls into smaller pieces is a time intensive and difficult procedure. The embodiments of the medical device discussed below can remove the tissue balls in one piece. In addition, the tissue removal procedure using the medical device may be less time-consuming as compared to conventional methods of removal discussed above.

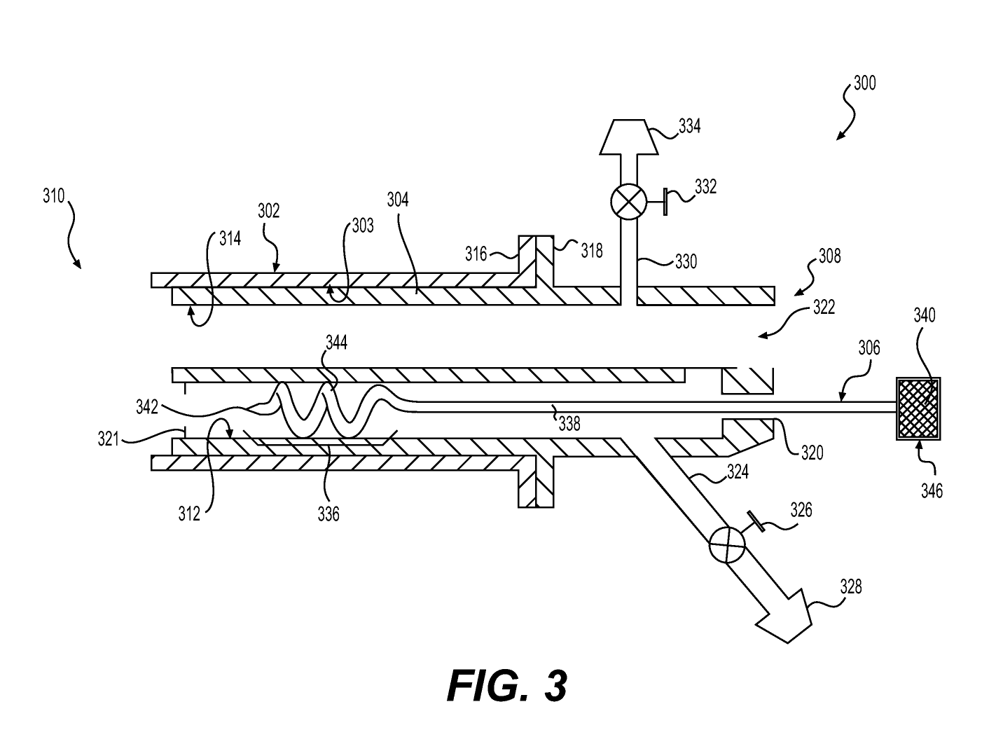

FIG. 3 illustrates an exemplary medical device 300 that may be used to remove large pieces of severed tissue, e.g., tissue balls, from a body cavity, according to embodiments of the present disclosure. The medical device 300 may include an outer sheath 302, which may include a working lumen 303 extending therethrough. The working lumen 303 may be configured to receive a multi-lumen element or sheath 304 and a working member 306 therein.

The outer sheath 302 may be a catheter, an endoscope, and/or any other appropriate luminal introducer structure configured to be inserted and manipulated within a patient's body. Particularly, the outer sheath 302 may be a cystoscope or a rectoscope that may be inserted into the urethra of a patient. The cystoscope, as is known in the art, is an endoscopic instrument that may be passed through the urethra for visualization and or manipulation of tissue. The outer diameter of the outer sheath 302 may range from about 1 Fr to about 100 Fr, preferably the outer diameter of the outer sheath 302 may range from about from about 10 Fr to about 50 Fr. In some embodiments, the outer diameter of the outer sheath 302 may range from about 20 Fr to about 28 Fr. Catheters, endoscopes, rectoscopes, and cytoscopes are well known in the prior art and thus, the outer sheath 302 will not be discussed in detail further.

The multi-lumen sheath 304 may include a proximal end 308 and a distal end 310. The multi-lumen sheath 304 may extend along a longitudinal axis and may include a first lumen 312 and a second lumen 314. The first and second lumens 312 and 314 may extend parallel to each other and the longitudinal axis of the multi-lumen sheath 304. Alternatively or additionally, the multi-lumen sheath 304 may include one or more additional lumens (not shown) through which an operator may introduce one or more medical devices. In an alternative embodiment, sheath 304 may include only one lumen.

The multi-lumen sheath 304 may have a substantially circular cross-sectional shape. Other suitable cross-sectional shapes such as elliptical, oval, polygonal, or irregular may also be used. In some embodiments, the diameter of the multi-lumen sheath 304 may range from about from about 10 Fr to about 50 Fr. In some embodiments, the diameter of the multi-lumen sheath 304 may range from about 20 Fr to about 28 Fr.

The outer sheath 302 and multi-lumen sheath 304 may connect to each other using one or more coupling mechanisms, such as, use of adhesives, clips, or flanges. In some embodiments, as shown, a flange 316 on the outer sheath 302 and a flange 318 on multi-lumen sheath 304 are used to secure the outer sheath 302 and the multi-lumen sheath 304 together. The flanges 316 and 318 may be affixed together with any appropriate mechanical and/or chemical connection, such as, adhesives, screws, bolts, pins, clamps, corresponding protrusions and indentations, or the like.

The first lumen 312 may be configured to receive the working member 306. As shown, a port 320 on the proximal end 308 of multi-lumen sheath 304 may be used for inserting the working member 306 into the first lumen 312. The port 320 may have any cross-sectional dimensions sufficient to allow axial and rotational motion of the working member 306. As shown in FIG. 3, the port 320 may have a narrower diameter than the first lumen 312. The narrower diameter of the port 320 may support the working member 306 and prevent any undesired motion or displacement of working member 306.

In addition, the distal end of the first lumen 312 may contain one or more tissue holding feature such as a radially inward protrusion(s) 321 extending therein. The protrusion(s) 321 may act as a one-way stop valve for the captured tissue. The protrusion(s) 321 may be configured so as to bend in one direction so as to allow tissue to enter the first lumen 312 but may be rigid and prevent bending in a second direction, opposite the first direction so as to prevent the tissue from leaving the first lumen 312 after the tissue has entered the first lumen 312. In addition, the protrusion(s) 321 may prevent the tissue from rotating along with the working member 306, when the working member 306 pierces into the tissue.

In an embodiment, for example, the protrusion(s) 321 may be comprised of a directionally biased material or structure in the form of a ring or band attached to the inner diameter of the first lumen 312, which bends when the captured tissue is pulled in, but becomes rigid if the tissue tries to escape the first lumen 312. For example, as tissue is pulled into the first lumen 312, the protrusion(s) 321 may bend such that the radially inner most end of the protrusion(s) 321 moves in a proximal direction (e.g., toward the right side of FIG. 3) so as to permit the tissue to be received within the first lumen 312. If, however, the tissue received within the first lumen 312 receives a distally directed force (e.g., a pulling or pushing force urging the tissue toward the left side of FIG. 3), the protrusion(s) 321 may return towards its original orientation as shown in FIG. 3. As such, the end of the protrusion(s) 321 may straighten such that the protrusion(s) 321 is no longer bent. Accordingly, tissue received within the first lumen 312 is prevented from moving distally outward of the first lumen 312 via the protrusion(s). In this embodiment, the protrusion(s) 321 may grip the captured tissue firmly to prevent it from rotating along with the working member 306. Alternatively, in some other embodiments, the protrusion(s) 321 may take a different form, such as disks, rims, a male thread, hooks, or pins, to prevent the tissue from leaving the first lumen 312 and rotating along with the working member 306. In an alternate embodiment, the tissue holding feature may instead be in the form of a valve, such as a film over the distal end of first lumen 312. The film may include a hole or slot therethrough or a proximally facing cone configured to permit tissue into the lumen, but to prevent tissue received within the first lumen 312 from moving distally outward of the first lumen 312.

Further, the first lumen 312 may be in fluid communication with a suction line 324 including a suction valve 326 and a coupling 328. The suction line 324 may facilitate removal of tissue from the target location. The suction line 324 may create a vacuum in the first lumen 312 to urge or draw the tissue excised by the HoLEP procedure in the first lumen 312. The suction line 324 may be coupled to an external vacuum pump at the coupling 328. In addition, the suction pressure may be controlled by the suction valve 326. Further, the port 320 may have a rubber or polymeric seal to prevent contamination and loss of suction in the first lumen 312.

The multi-lumen sheath 304 may include one or more optical components (not shown) positioned in second lumen 314. For example, such optical components may include telescopic optics, lenses, mirrors, a digital camera, an eye piece, or other optical components arranged within the second lumen 314. By way of example only, an eye piece or viewing window (not shown) may be positioned within a port 322 positioned at the proximal end 308 of multi-lumen sheath 304 and in communication with the second lumen 314. The optical components may be connected to a remote imaging system for displaying and/or recording images captured by the optical components. For example, the optical components may include one or more fiber optic cables for transmitting light to the distal end 310 of the multi-lumen sheath 304, and transmitting reflected images from the distal end 310 to the proximal end 308 of the multi-lumen sheath 304. Other optical components may be included, including those which are configured to work with a digital camera including processing components, conductors and/or data transmission systems. Further, a suitable light source may be utilized for providing illumination to visualize a target location with the help of the optical components, e.g., xenon light, LEDs, or other suitable light sources.

In addition, the second lumen 314 may be in fluid communication with an irrigation line 330 including a valve 332 and a coupling 334. The irrigation line 330 may be used to deliver a fluid, such as, water or saline solution to a target location within the body. For example, the fluid may be introduced into the bladder to help protect the tissue wall or mucosal lining of the bladder, for example, from damage during a medical procedure. The fluid may include a hemostatic agent configured to help slow bleeding. Other agents may also be included in a fluid that promote healing of the resected organ. The fluid may be adjusted for temperature, such as heated fluid, to promote hemostasis. Further, the irrigation line 330 may facilitate cleaning of blood or tissue pieces that may become lodged on one or more of the optical components. The irrigation line 330 may be connected to an external irrigation source or reservoir for delivering or injecting the fluid through the second lumen 314 into the bladder (not shown). An external fluid system may be coupled to the irrigation line at the coupling 334. The valve 332 may be used to control the flow of fluid into the second lumen 314.

As shown in FIG. 3, the working member 306 may be an elongate member having a distal portion 336, a shaft 338, and a proximal handle 340. The working member 306 may be capable of rotating about a longitudinal axis of and within the first lumen 312. In some embodiments, the working member 306 may also be able to move laterally back-and-forth along the longitudinal axis of the first lumen 312.

The distal portion 336 may be configured as an auger (e.g., corkscrew) portion and as such, may include an auger or a gimlet. The distal portion 336 may include a helical tool configured to be rotated by a handle. For example, the distal portion 336 may include a plurality of helical turns 344. Even so, the working element may comprise an auger with only one helical turn or less than one helical turn (e.g., a portion of one helical turn). It is to be noted that any number of helical turns 344 may be employed. Additionally, the distal portion 336 may include a sharpened tip 342. The sharpened tip 342 may be configured to assist in piercing tissue, e.g., a tissue ball formed by an enucleating procedure. During use, an operator may apply suction to a tissue ball via suction line 324. As such, a portion of tissue ball 324 may be urged (e.g., sucked) into the first lumen 312. Next, an operator may position the distal portion 336 proximate the tissue ball. After positioning, the operator may pierce the tissue ball with the sharpened tip 342 and rotate the distal portion 336 so as to urge or draw the tissue ball into the plurality of helical turns 344. Such rotation may embed the plurality of helical turns 344 into the tissue ball such that subsequent movement of the distal portion 336 may cause similar movement to the tissue ball. For example, upon embedding the plurality of helical turns 344 into the tissue ball, applying a proximally-directed axial force (e.g., pulling force) to the distal portion 336 may result in a corresponding proximally directed axial movement of the tissue ball such that the tissue ball is captured, lodged, or otherwise received, either partially or completely, within the first lumen 312. In one embodiment, it is understood that the tissue ball may be drawn (e.g., sucked) into the first lumen 312 with sufficient force such that no distally-directed axial movement (e.g., pushing) of the distal portion 336 is necessary to cause sharpened tip 342 to pierce the tissue ball. In other embodiments, it is understood that some distally-directed axial movement (e.g., pushing) of the distal portion 336 may be necessary to pierce the tissue ball with the sharpened tip 342. As such, the distal portion 336 may be configured to pierce and embed within excised tissue by application of one or both of forward pushing and rotational force.

As described above, the helical turns 344 may facilitate in pulling the tissue ball into the first lumen 312. Accordingly, in some embodiments, a cross-sectional shape of a wire forming the plurality of helical turns 344 may include a sharpened edge (not shown). The sharpened edge or ridge may be configured to cooperate, grip, or otherwise interact with the tissue ball so to assist in embedding the auger or corkscrew portion within the tissue ball. It is understood, however, that any cross-sectional wire shape may be used, for example, circular, square, rectangular, ovular, and the like. Helical turns may have any pitch, angle or diameter.

In an alternative embodiment, distal portion 336 may be formed as a spear tip, arrow tip, barbed tip or other embodiments designed to pierce and hold tissue. Such embodiments allow for stacking of tissue on working member 306. Further, such embodiments do not require rotation to operate.

The distal portion 336 may be sufficiently rigid and/or stiff so as to bore into the tissue. Thus, distal portion 336 may be made from a biocompatible rigid material, such as, Nitinol™, Stainless Steel, alloys, and/or HDPE.

In some other embodiments, the distal portion 336 may be a straight rod-like structure made of a superelastic material or shape memory material such as, Nitinol™, having a piercing tip. The distal portion 336 may be transitioned into an auger shape, similar to the embodiments described above, by actuation, after it pierces the desired tissue. For example, the distal portion 336 may be actuated by application of heat or electric current. This embodiment may reduce the pre-operation profile of the distal portion 336 within the first lumen 312. Alternatively, the distal portion 336 may be configured to translate into any other shape that may be suitable to engage the excised tissue and pull it into the first lumen 312.

The shaft 338 may be an elongate member connecting the distal portion 336 to the proximal handle 340. The shaft 338 may comprise one or more flexible parts such that at least a portion of the shaft 338 has a flexible or a semi-flexible structure. The shaft 338 may be flexible along its entire length or along at least a portion of its length. In some embodiments, the proximal portion of the shaft 338 may be flexible, while the distal portion may be less flexible—relatively rigid or non-flexible. Flexibility of the proximal portion may help the shaft 338 maneuver twists and turns in body lumens and/or deflect in the desired direction, and allow insertion of the working member 306 into the first lumen 312 through entry ports 320 set at an angular position with respect to the longitudinal axis of the first lumen 312. For example, in some embodiments, the entry port 320 may be set at an angle (e.g., 45°) to the first lumen 312. Rigidity of the distal portion may provide the necessary force to rotate and/or push the working member 306 forward and may assist in piercing tissue. The shaft 338 may have any cross-sectional shape, such as circular, oval, polygonal, or irregular. Additionally, any one or more portions of the shaft may be reinforced by, for example, filament braiding located in the wall of the shaft. Portions of the shaft may be weakened with a less rigid material and/or by the lack of reinforcement, and, for example, with grooves, holes or the like.

A portion of the shaft 338 and the handle 340 may extend proximally out of the multi-lumen sheath 304 via port 320. The handle 340 may include and/or may be shaped like a knob to facilitate rotation of the working member 306. The handle 340 may be temporarily or permanently coupled to the proximal end of the shaft 338 using techniques known in the art such as, e.g., via welding, adhesive, or other attachment, or may be integrally formed from a single continuous piece of material with the shaft 338. In some embodiments, the handle 340 may include a grip coating 346 to facilitate handling. Alternatively, handle 340 may include a frictional element such as knurled or otherwise textured surface to aid the user to grip the working members 306. The frictional element may further comprise an elastomer or other grippable polymer coating or overlay.

The multi-lumen sheath 304 and the working member 306 may comprise one or more suitable biocompatible materials, including rigid, flexible, and/or semi-rigid materials. Exemplary materials include, but are not limited to, polymers, metals and metal alloys, rubber, silicone, and metal-polymer composites. Additionally, the multi-lumen sheath 304 and the working member 306 may be made from the same material(s) or from different material(s).

In some embodiments, the distal portion 336 of working member 306 may be made of biocompatible shape memory alloys or polymers that may be actuated through electrical or thermal stimulus. The shaft 338 may be made of a mono- or multi-filament polymeric or metallic shaft with a decreasing stiffness gradient from the distal to the proximal end of the shaft 338. Alternatively, the shaft 338 may include a metallic or polymeric rigid distal shaft and a metallic or polymeric flexible proximal shaft coupled together at a junction positioned appropriately along the length of the shaft 338. The handle 340 may be constructed from any suitable materials such as metal, polymeric, and/or ceramic materials.

The outer surface of the multi-lumen sheath 304 may include a suitable coating, e.g., a hydrophilic coating, to aid in smooth insertion and/or removal of the multi-lumen sheath 304 within the outer sheath 302. Similarly, the working member 306 may include one or more coatings. For example, suitable low-friction material, such as polytetrafluoroethylene (PTFE), polyetheretherketone (PEEK), polyimide, nylon, polyethylene, or other lubricious polymer coatings may be applied to one or more components of the working member 306, the distal portion 336 and/or shaft 338 for ease of motion of the working member 306 in the first lumen 312.

The following describes an exemplary method for removing excised tissue from a body according to the present disclosure. The method may be utilized for removing the excised tissue from patient's bladder, however, it is understood that the method may be utilized in other parts of the body. The medical device 300 may be introduced through the urethra of the patient and may be advanced toward the bladder. The method may further include rotating the distal portion 336 so as to proximally urge the target tissue into the first lumen 312. In this process the plurality of helical turns 344 of the distal portion 336 may embed themselves into the target tissue. Additionally, the working member 306 may be rotated and/or pushed distally with the help of the handle 340 (knob) such that the distal portion 336 pierces the target tissue. Suction may be applied within the first lumen 312 so as to pull the tissue within the first lumen 312. Then, the working member 306 may be pulled proximally within first lumen 312 thereby pulling the target tissue further into the first lumen 312. Once the tissue is pulled into the first lumen 312, the protrusion(s) 321 may prevent the captured tissue from rotating along with the working member 306 and moving out of the second lumen 314. Finally, the operator may retract the multi-lumen sheath 304 from the patient's body removing the excised tissue lodged within it.

Using the exemplary methods described above, the medical device 300 may be used for extraction of resected tissue such as lesions from a patient's body for treatment or diagnostic purposes. The medical device 300 may be a single-use device which can be discarded after one use or may be used again after sterilization.

Other embodiments of the present disclosure will be apparent to those skilled in the art after consideration of the specification and practice of the embodiments disclosed herein. It is intended that the specification and examples be considered as exemplary only, with a true scope and spirit of the disclosure being indicated by the following claims.

What is claimed is:

1. A medical device, comprising:
an elongate luminal element configured for insertion into a natural body lumen;
an elongate working member positioned within the elongate luminal element, the elongate luminal element including at least a first lumen extending along a longitudinal axis, the working member including an auger portion on a distal end thereof, the auger portion being configured to pierce tissue; and
wherein the auger portion is rotatable about the longitudinal axis.

2. The medical device of claim 1, further including:
a suction line in fluid communication with the first lumen, the suction line configured to urge tissue into the first lumen.

3. The medical device of claim 1, wherein the elongate luminal element includes a second lumen, the second lumen configured to receive an optical member therein.

4. The medical device of claim 3, further including:
an irrigation line in fluid communication with the second lumen.

5. The medical device of claim 1, wherein the elongate member includes a first portion and a second portion, and wherein the first portion is more flexible than the second portion.

6. The medical device of claim 1, wherein the elongate member includes a handle portion, the handle portion including a grip thereon.

7. The medical device of claim 1, wherein the first lumen further includes a tissue holder at a distal portion thereof, the tissue holder extending radially inwardly toward the longitudinal axis.

8. The medical device of claim 1, wherein the auger portion includes at least a portion of a helical turn.

9. The medical device of claim 1, wherein the auger portion includes a sharpened edge configured to pierce tissue.

10. The medical device of claim 1, further including:
at least one of a rectoscope or cystoscope configured to receive the elongate luminal element therein.

11. A method of removing tissue from a body of a patient, the method comprising:
inserting a medical device proximate to a target tissue, the medical device including an elongate working member configured to be received with a first lumen of an elongate luminal element, the first lumen extending along a longitudinal axis, the working member including an auger portion on a distal end thereof;
piercing the target tissue region with the auger portion;
rotating the auger portion so as to proximally urge the target tissue into the first lumen; and
removing the medical device from the body of the patient.

12. The method of claim 11, further including:
applying a suction force to the first lumen so as to urge tissue into the first lumen.

13. The method of claim 11, wherein the first lumen further includes a tissue holder at a distal portion thereof, the tissue holder extending radially inwardly toward the longitudinal axis and configured to assist in preventing tissue pierced by the auger portion from rotating within the first lumen.

14. The method of claim 11, wherein the auger portion includes at least a portion of a helical turn.

15. The method of claim 11, wherein the elongate luminal element includes a second lumen, the second lumen configured to receive an optical member therein, the method further comprising:
irrigating the second lumen with an irrigation source.

16. A medical device, comprising:
a delivery member configured for insertion into a natural body lumen;
a working member configured to be received within a first lumen of the delivery member, the first lumen extending along a longitudinal axis, the working member including an auger portion on a distal end thereof, the auger portion including a sharpened distal tip configured to pierce tissue; and
wherein the auger portion is rotatable about the longitudinal axis.

17. The medical device of claim 16, further including:
a suction line in fluid communication with the first lumen, the suction line configured to urge tissue into the first lumen.

18. The medical device of claim 16, wherein the auger portion includes a sharpened edge configured to urge tissue into the first lumen when the auger portion is rotated.

19. The medical device of claim 16, wherein the first lumen further includes a tissue holder at a distal portion thereof, the tissue holder extending radially inwardly toward the longitudinal axis.

20. The medical device of claim 16, wherein the delivery member further includes a second lumen, the second lumen configured to receive an optical member therein, the device further including:
an irrigation line in fluid communication with the second lumen.
